(19) United States
(12) Patent Application Publication
(10) Pub. No.: US 2015/0081018 A1
(43) Pub. Date: Mar. 19, 2015

(54) METHODS AND SUBSTRATES FOR DIFFERENTIATION OF NEURAL STEM CELLS

(71) Applicant: Rutgers, The State University of New Jersey, New Brunswick, NJ (US)

(72) Inventors: Ki-Bum Lee, Monmouth Junction, NJ (US); Aniruddh Solanki, South Plainfield, NJ (US); Shreyas Shah, Dayton, NJ (US)

(21) Appl. No.: 14/464,183

(22) Filed: Aug. 20, 2014

Related U.S. Application Data
(63) Continuation of application No. 13/214,824, filed on Aug. 22, 2011, now abandoned.
(60) Provisional application No. 61/379,405, filed on Sep. 2, 2010.
(60) Provisional application No. 61/375,627, filed on Aug. 20, 2010.

(51) Int. Cl. A61L 27/54; C12N 5/0793

(52) U.S. Cl. CPC A61L 27/54; C12N 5/0619; C12N 2506/08; C12N 2533/50; C12N 2535/10; A61L 2300/608
USPC 623/11.11; 435/377; 435/366

(57) ABSTRACT

The present invention is directed to methods and substrates for promoting the differentiation of neural stem cells to neurons.

### CROSS REFERENCE TO RELATED APPLICATIONS

This application claims the benefit of U.S. Provisional Application Nos. 61/375,627 filed Aug. 20, 2010 and 61/379,405 filed Sep. 2, 2010, the disclosures of which are incorporated herein in their entireties.

### STATEMENT REGARDING FEDERALLY SPONSORED RESEARCH

This invention was made, at least in part, with government support under Director's Innovator Award No. 1DP20D006462-01 awarded by the National Institutes of Health. The U.S. government has certain rights in the invention.

### BACKGROUND OF THE INVENTION

The ability of stem cells to differentiate into specialized lineages within a specific microenvironment is vital for regenerative medicine. Neural stem cells (NSCs) are multipotent and differentiate into neurons and glial cells (Gage et al. (2000) Science 287:1433), which can provide essential sources of engraftable neural cells for devastating diseases such as Alzheimer's disease, Parkinson's disease and spinal cord injury. One of the challenges involved in the differentiation of NSCs is the identification and optimization of factors that result in an increased proportion of NSCs differentiating into neurons as opposed to glial cells. Soluble cues such as brain-derived neurotrophic factor (BDNF), sonic hedgehog (Shh), retinoic acid (RA), and neuropathiazol have been shown to significantly increase neuronal differentiation of NSCs in vitro (Bath et al. (2010) Developmental Neurobiology 70:339; Li et al. (2009) Development 136:4055; Song et al. (2002) Nature Neuroscience 5:438; Warashina et al. (2006) Angewandte Chemie-international Edition 45:591). However, the research toward studying the function of two other microenvironmental cues (cell-cell interactions and insoluble cues) during the neuro-differentiation of NSCs is limited. While various aspects such as cell-cell interactions, combinations of extracellular matrix (ECM) proteins, and physical properties of substrates have been shown to play a role in determining the fate of other adult stem cells such as mesenchymal stem cells (MSCs), cardiac stem cells, and hematopoetic stem cells, (Park et al. (2007) Advanced Materials 19:2530; Forte et al. (2008) Stem Cells 26:2093; Taichman (2005) Blood 105:2631), little is known about the influence of such factors on the neuronal differentiation of NSCs.

### SUMMARY OF THE INVENTION

The present invention, in one embodiment, provides substrates having one or more extracellular matrix proteins disposed thereon in geometric patterns and dimensions suitable to promote the differentiation of neural stem cells (NSCs) to neurons. In one embodiment, the substrate is a biocompatible substrate. In another embodiment, the substrate has at least one NSC in contact therewith.

In another embodiment, the present invention provides methods for promoting the differentiation of NSCs to neurons comprising contacting NSCs with a substrate having one or more extracellular matrix proteins disposed thereon in geometric patterns and dimensions suitable to promote the differentiation of NSCs to neurons.

In another embodiment, the present invention provides a biocompatible implant comprising a substrate having one or more extracellular matrix proteins disposed thereon in geometric patterns and dimensions suitable to promote the differentiation of neural stem cells to neurons. In another embodiment, the implant further comprises at least one NSC.

In another embodiment, the present invention provides a method of treating or ameliorating a neurodegenerative disorder or a neurological injury comprising administering the biocompatible implant of the present invention to a subject in need of such treatment.

The present invention provides, in another embodiment, compositions and kits comprising the substrates of the invention.

### BRIEF DESCRIPTION OF THE DRAWINGS

FIGS. 1A-D are schematic diagrams showing (A) the fabrication of ECM protein patterns and their application for NSC differentiation; (B) the selective attachment of NSCs on the protein patterns and differentiation into two different kinds of neural cells; (C) the differentiation of NSCs into either neurons or astrocytes on the protein patterns; and (D) increased neuronal differentiation on the grid patterns, as compared to the stripes and squares.

FIGS. 2A1-C3 show growth and differentiation of NSCs on laminin patterns. Phase contrast images show NSC attachment and growth on stripes (A1), squares (B1), and grids (C1) on Day 2 after seeding. Fluorescent images of cells stained for the neuronal marker TuJ1 and nucleus show the extent of neuronal differentiation on stripes (A2), squares (B2), and grids (C2) on Day 6 after seeding. Cells stained for astrocyte marker GFAP and nucleus show the extent of glial differentiation on stripes (A3), squares (B3), and grids (C3) on Day 6 after seeding. Scale bars: 50 μm

FIG. 3 depicts a quantitative comparison of the percentage of cells expressing the neuronal marker TuJ1 and the astrocyte marker GFAP on laminin patterns of squares, stripes and grids. Six days after seeding, the differentiated cells were counted and plotted as a ratio of TuJ1-positive cells or GFAP-positive cells to the total number of cells (n=3). Student's unpaired t-test was used for evaluating the statistical significance for cells stained for TuJ1 on stripes and squares, compared to those on grids. (*=P<0.01, **=P≦0.001)

FIGS. 4A-D show NSC alignment and differentiation on combinatorial ECM patterns. (A) NSCs on grids of laminin express the neural stem cell marker, nestin (purple) on Day 2 after seeding, thus confirming that the NSCs are undifferentiated. (B) NSCs stained for actin (green) show extensive spreading and cell-cell interactions on grid patterns of laminin on Day 2 after seeding, confirming that the NSCs, while still in the undifferentiated state, extensively interact with each other. (C) SEM image of NSCs on Day 2 after seeding, showing the early alignment and extension of processes on grid patterns of laminin. (D) NSCs previously shown to extend and grow on the grid patterns of laminin undergo neuronal differentiation and express the neuronal marker synapsin (pseudocolored yellow) on Day 6 after seeding. Scale bars: 20 μm

FIGS. 5A-C show immunostaining with anti-laminin IgG. The laminin patterns generated were confirmed using anti-laminin IgG (Sigma). Consistent results were obtained and reproduced for the three different geometries (A) stripes, (B) squares, and (C) grids having varying dimensions. Scale bars: 20 μm

FIGS. 6A-D show NSC alignment and differentiation on combinatorial ECM protein arrays. NSCs on stripes (A) and squares (C) of laminin express the neural stem cell marker, nestin (purple) on Day 2 after seeding, thus confirming that the NSCs are undifferentiated. NSCs stained for actin (green) on stripes (B) and squares (D) of laminin on Day 2 after seeding. Scale bars: 20 μm

FIGS. 7A-C show colocalization of TuJ1 and synapsin within cells on grid patterns. (A) TuJ1; (B) synapsin; (C) merged image showing the overlap of the two markers. Scale bar: 20 μm

FIG. 8 shows an FESEM image of NSCs on grids of laminin on Day 2 after seeding, and shows the early alignment and extension of processes on grid patterns of laminin. Scale bar: 20 μm

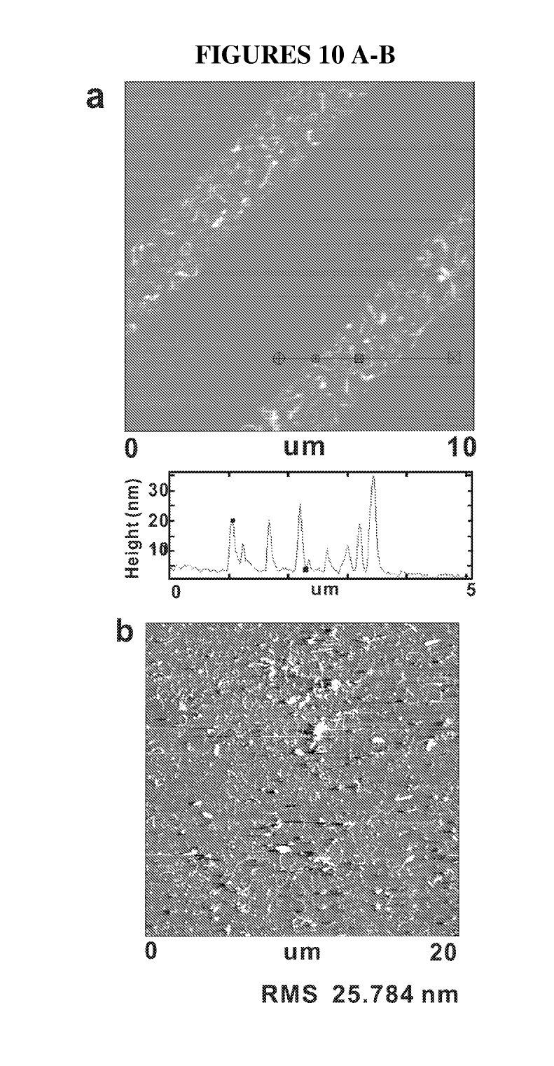

FIGS. 9A-D depict CNT network patterns for selective hNSC growth and polarization. (A) Schematic diagram showing polarization-controlled neural differentiation using CNT patterns. CNT monolayer patterns were fabricated on a substrate, and laminin was absorbed selectively on the CNT-coated regions. This structure induced preferential adhesion of hNSCs, finally achieving polarization-controlled neuronal differentiation. (B) SEM image of CNT patterns (dark spots). Scale bar represents 40 μm. (C) Immunofluorescence image of anti-laminin (green) bound to the laminin which was selectively adsorbed on the CNT patterns. The scale bar represents 200 μm. It confirms the selective adsorption of laminin on CNT. The insert shows the AFM topography image of the laminin-coated CNT monolayer in phosphate buffered saline (PBS). The scale bar in the insert represents 2 μm. (D) Cell viability assay of hNSCs on CNT patterns for three-day proliferation. The viability was measured by flow cytometry. The obtained data in the graph clearly indicate that 98% of hNSCs grown on the CNT layer were alive.

FIGS. 10A and B depict monolayer topography. (A) depicts AFM topography image of multi-walled CNT monolayer patterns created on the Au substrate. As indicated in the height profile (lower), the height of the CNT monolayer nanostructure ranged from 10 to 40 nm. (B) depicts topography of laminin-coated CNT monolayer structures in PBS. The average surface roughness of the laminin-coated CNT networks measured by AFM was 25.784 nm.

FIGS. 11A-F depict a cell viability assay by flow cytometry of hNSCs on CNT pattern: three-day proliferation and three-differentiation, respectively. Cell viability assay was carried out by flow cytometry. It was measured by the calculations performed automatically (Guava ViaCount, Millipore). The acquired data are displayed in dot plot: viability (PM1) and nucleated cells (PM2). Live cells appear on the left side of the plot. Dead cells are on the right side of the plot. Apoptotic cells are shown in between the live and dead cell populations. Viability data on (A) Control group grown on laminin-coated Au surface for three days (Control), (B) CNT pattern (5 μm×5 μm, 5-μm spacing) for three day proliferation (CNT-5 μm-D3), (C) CNT pattern (5 μm×5 μm, 5-μm spacing) for three day differentiation (CNT-5 μm-DF3), (D) CNT pattern (200 μm×200 μm, 200-μm spacing) for three day proliferation (CNT-200 μm-D3), (E) CNT pattern (200 μm×200 μm, 200-μm spacing) for three day differentiation (CNT-200 μm-DF3). (F) The cell viability data (%) obtained from each sample is shown in the graph.

FIGS. 12A and B depict Western Blot analysis of hNSCs. Nestin (monoclonal, 1:5000, Millipore, Temecula, Calif., USA) and SOX2 (monoclonal, 1:2500, Millipore, Temecula, Calif., USA) were used to confirm undifferentiated human neural stem cells (hNSCs). GFAP (monoclonal, 1:2500, Millipore, Temecula, Calif., USA) and TUJ1 (monoclonal, 1:1000, Millipore, Temecula, Calif., USA) were used to confirm the differentiation into glial cells and neurons, respectively. NIH3T3 cells were used as a negative control. hNSCs were cultured and differentiated on laminin-coated cell culture petri dishes. (A) Western blot results of hNSCs grown in culture media with the growth factors (EGF and bFGF). The cells exhibited positive for nestin and SOX2, indicating undifferentiated hNSCs. (B) Western blot results of hNSCs grown in culture media without the growth factors. The cells were positive for GFAP and TUJ1, indicating the differentiation of hNSCs (D-hNSC) into neuronal cells.

FIG. 13 depicts the hNSC seeded on CNT square pattern (100-μm-width, 200-μm-spacing). The rapidly attached hNSCs on the CNT-coated regions (black dotted square) were observed within 30 min after cell seeding as shown in dark gray color, while those on ODT SAM area in white circular shapes were not adhered yet. Scale bar represents 200 μm.

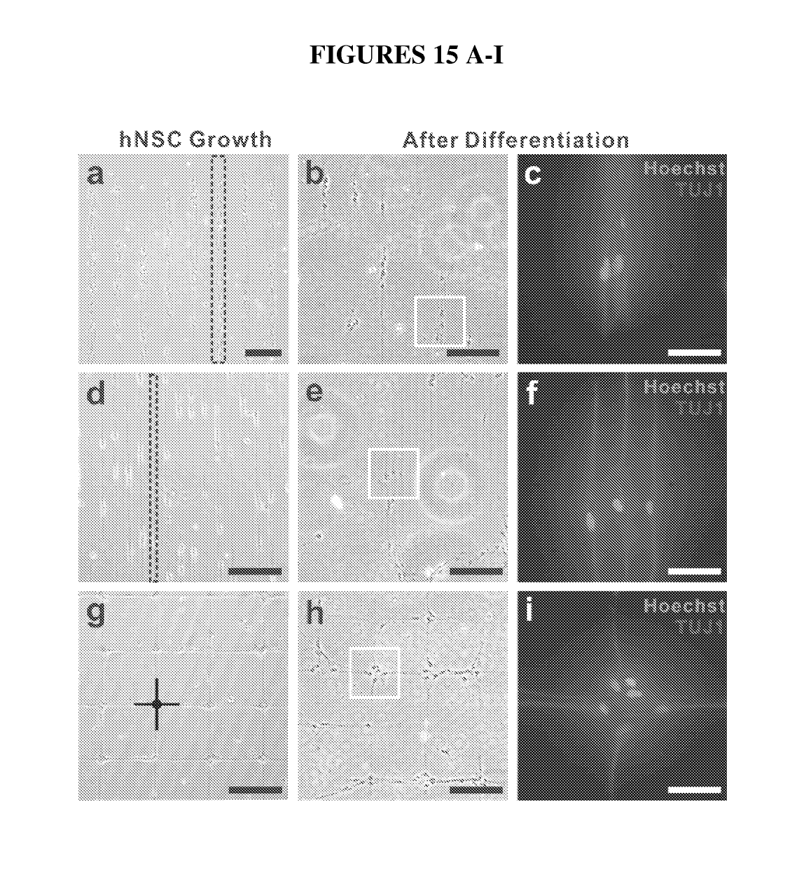

FIGS. 14A-I show hNSC growth and differentiation depending on the size of CNT patterns. The phase contrast images of hNSCs grown for 1 day (A,D,G) and those of the differentiated cells for 2 weeks (B,E,H), and the immunofluorescence images of the differentiated cells (C,F,) are shown. The immunofluorescene markers are Hoechst for nuclei, glial fibrillary acidic protein (GFAP) for astroglial cells, and TUJ1 and neurofilament light (NF-L) for neuronal cells. All scale bars represent 200 μm, unless otherwise noted. The dotted black squares (A,B,D,E) indicate some of the CNT-coated regions. (A-C) hNSC growth and differentiation on rather large square-shape CNT patterns (300 μm×300 μm, 200 μm spacing). Neural networks were constructed in arbitrary manner after differentiation. The immunofluorescence image (C) shows the differentiated cells positive for the astroglial marker, GFAP (green). (D-F) Restrictive neurite growth of hNSCs in individual CNT square patterns (50 μm×50 μm, 50 μm spacing). No indication of neurite outgrowth of hNSC was observed after the growth and differentiation from the immunostaining image of NF-L (red). (G−1) Outgrowths of hNSCs directed by rather small square-shape CNT patterns (5 μm×5 μm, 5 μm spacing). The inset figure (G) shows that a single hNSC was attached on seven individual CNT square patterns. The immunofluorescence image (I) indicates that the differentiated cells are positive for neuronal cell marker, TUJ1 (red). The scale bar in the phase contrast image (G) represents 100 μm.

FIGS. 15A-I depict control of hNSC orientation using line shape CNT patterns. (A-C) hNSC growth on CNT line shape patterns (30 μm width). The hNSCs inside individual 30 μm wide line patterns were observed to grow, extending their neurites in the same direction along the predefined CNT line patterns. (D-F) Individual hNSC growth on each CNT line pattern (5 μm width). The hNSCs were aligned to form a bipolar shape on the CNT line patterns during the growth and differentiation. (G-I) Neural network formed on narrow line shape CNT patterns combined with large square-shape ones. Highly oriented hNSC growth was induced by the predefined CNT patterns, and eventually well-organized neural networks were formed after differentiation. (A,D,G) Phase contrast images of hNSC grown for 1 day, and the scale bars in the phase contrast images are 200 μm. (B,E,H) Phase contrast images of the differentiated cell. (C,F,L) Immunofluorescence images of the differentiated cells. The scale bars in the phase contrast images are 50 μm. The dotted black squares indicate some of the CNT-coated regions.

FIG. 16 depicts long-term differentiated hNSCs on CNT line patterns (100-μm-width, 200-μm spacing) for four weeks. The dark regions of the phase contrast image indicate the CNT line patterns. Scale bar represents 200 μm.

FIG. 17 depicts phase contrast image (left first) and immunofluorescence images of the differentiated hNSCs on CNT patterns. Scale bars represent 200 μm. The CNT patterns are comprised of square-shape patterns connected by narrow line-shape patterns. Hoechst and synaptophysin were used as immunostaining markers for nuclei and neuronal presynaptic vesicles, respectively. The differentiated hNSCs formed neural network on the CNT patterns with synapse formation (green color from the merged image).

FIGS. 18A-D depict control of hNSC growth and differentiation on biocompatible and flexible polyimide (PI) substrate. (A) Optical image of a polyimide membrane with CNT patterns which is flexible and transparent. (B) Immunofluorescence image of anti-laminin (green). It confirms that the laminin was selectively adsorbed onto the CNT patterns on PI substrate. Scale bar represents 200 μm. (C) Phase contrast image of selective hNSC adhesion on CNT patterns on PI after cell seeding. Scale bar represents 200 μm. (D) Immunofluorescence image of the differentiated hNSCs on CNT patterns on PI (TUJ1 for neural cells and GFAP for astroglial cells). The insert shows the magnified image of the region marked by the white solid square. Scale bar represents 200 μm and that of the insert represents 50 μm. The orientation-controlled neural networks were constructed along the CNT patterns on PI membrane.

FIGS. 19A and B depict CNT patterns on flexible polyimide (PI) substrate and hNSC differentiation for three weeks. (A) SEM image of CNT patterns fabricated on PI substrate. Scale bar represents 50 μm. (B) Phase contrast image (left) and immunofluorescence image (right) of the differentiated hNSCs after three-week differentiation. Immunofluorescence images are DAPI for nucleus (blue), GFAP for astrocytes (green) and TUJ1 for neuron (red). Scale bar represents 200 μm.

FIGS. 20A-E depict structural-polarization-controlled neuronal differentiation of individual hNSCs using CNT patterns. (A) SEM image of a CNT pattern with a single narrow strip as shown in dark gray. (B) Phase contrast images of hNSC adhesion on CNT patterns. The dotted square and line (red) represent the CNT patterns. Scale bar represents 50 μm. After cell seeding, the cell bodies of hNSCs were attached within the CNT square region. (C) Phase contrast images of the differentiated cells on the CNT patterns. The growing parts in the hNSCs were observed along the CNT single narrow strip regions during the differentiation. (D) Immunofluorescence images of growth-associated protein 43 (GAP 43, green) and Hoechst (blue, for nucleus). Scale bar represents 50 μm. The GAP 43 (green dots) was distributed along the narrow strip region. (E) Immunofluorescence image of GFAP (green), TUJ1 (red), and Hoechst (blue). Scale bar represents 50 μm. The differentiated neuronal cells (TUJ1, red) were surrounded by astroglial cells (GFAP, green) on the structural-polarization controlled CNT pattern, where the neuronal polarization was also directed by the CNT narrow strip region.

FIGS. 21A-D depict CNT bipolar patterns for axonal guidance and arborization. The markers are TUJ1 (red) for neuronal marker, GAP43 (green) for growth cone marker and Hoechst (blue) for nucleus in the immunostaining images. (A) Schematic of CNT bipolar patterns composed of circle array connected with narrow strips, which can enclose only single cell body within a circle. (B) Phase contrast image of hNSCs with the axon-like parts growing along the predefined C(NT strip pattern. Scale bar represents 200 μm. (C) Immunofluorescence image of the differentiated hNSCs on the bipolar pattern. Scale bar represents 200 μm. (D) Magnified image of immunofluorescence image of highlighted in (C), which showing clearly positive for the growth cone marker, GAP 43 (green). Growth cone of hNSC is clearly shown to be arborizing on the CNT circle pattern region after outgrowing along the protruding CNT strip pattern. Scale bar represents 50 μm.

FIGS. 22A-E show calculation of a neural-lineage percentage of the differentiated hNSCs on CNT polarization-control pattern. The number of immunoreactive cells for GFAP (astroglia) and TUJ1 (neurons) were analyzed and calculated using ImageJ (NIH). Each numbered sample indicates each nucleus region of individual cell from the immunofluorescence image. (A) Original fluorescence images of GFAP, TUJ1 and Hoechst (nuclei). The scale bar represents 50 μm. (B) Hoechst masking to quantify the corresponding nuclear regions of each marker of GFAP and TUJ1. The background signal of each image was subtracted before the masking processes. (C) Fluorescence signal intensities were presented as arbitrary unit and the intensities above a mean value in each marker were counted as ‘1’. (D) The table summarizing the image analysis. Only when one marker is positive and the other marker is negative, was the marker counted as immunoreactive. For example, if TUJ1=1 and GFAP=0, this is TUJ1 positive; if TUJ1=0, and GFAP=1, this is GFAP positive; if TUJ1=1, and GFAP=1, this might be called as ‘astron’ but it cannot be confirmed conclusively from this data; if TUJ1=0, and GFAP=0, this cell is not immunoreactive for the both markers at all. (E) The calculation result showed that 20% of the five hNSCs on the CNT polarization-control pattern were differentiated to neural cells and 20% of them to astroglial cells. The data was obtained from three different samples.

### DETAILED DESCRIPTION OF THE INVENTION

The present invention is based, in part, on the discovery that combinatorial patterns of extracellular matrix proteins having defined geometries and dimensions provide a microenvironment that may be used to regulate stem cell differentiation. Accordingly, in one embodiment, the present invention provides substrates having one or more extracellular matrix proteins disposed thereon in geometric patterns and dimensions suitable to promote the differentiation of neural stem cells (NSCs). Extracellular matrix proteins are known in the art and include, e.g. laminin, fibronectin, and collagens. In one embodiment of the present invention, the extracellular matrix protein is one or more of laminin, fibronectin, and collagen. In another embodiment, the extracellular matrix protein is laminin.

The geometric patterns of extracellular matrix proteins suitable to promote the differentiation of NSCs to neurons in accordance with the present invention include grids, and regular two-dimensional patterns of stripes, squares, lines, squares connected by lines, and circles connected by lines. The grids preferably comprise vertical and horizontal lines of from 10 μm to 50 μm in width separated by spaces of from 20 μm to 100 μm, and more preferably comprise vertical and horizontal lines of from 20 μm to 40 μm in width, separated by spaces of from 40 μm to 80 μm. The stripes preferably have dimensions of from 3000 μm to 5000 μm in length by from 10 μm to 50 μm in width and are separated from each other by spaces of from 20 μm to 100 μm, and more preferably have dimensions of about 4000 μm in length by from 20 μm to 40 μm in width and are separated from each other by spaces of from 40 μm to 80 μm. The squares preferably have dimensions of from about 1 μm to about 500 μm in length and width separated by spaces of from about 1 μm to about 350 μm. In one preferred embodiment, the squares have dimensions of about 300 μm in length and width separated by spaces of about 200 μm. In another preferred embodiment, the squares have dimensions of about 5 μm in length and width separated by spaces of about 5 μm. The lines preferably have widths of from 5 μm to 200 μm separated by spaces of from 10 μm to 400 μm. In one preferred embodiment, the lines have widths of from about 5 μm to about 30 μm separated by spaces of about 10 μm to about 60 μm. The circles preferably have diameters of from 5 μm to 100 μm separated by spaces of 10 μm to 200 μm and more preferably have diameters of from 25 μm to 75 μm separated by spaces of 50 μm to 150 μm.

The geometric patterns of extracellular matrix proteins suitable to promote the differentiation of NSCs in accordance with the present invention preferably have average height/depth dimensions of from about 50 nm to about 200 nm, and more preferably from about 100 nm to about 150 nm. The height/depth dimensions may be determined by methods known in the art, including for example Atomic Force Microscopy (AFM) topological data (height profile).

Substrates suitable for use in accordance with the present invention include conventional cell culture materials such as glass for in vitro applications, and biocompatible materials including, for example, polyimide, polyamide, polycarbonate, and silicone for in vitro and in vivo applications. In a preferred embodiment the substrate is polyimide, for example a polyimide membrane. Substrates having one or more extracellular matrix proteins disposed thereon in geometric patterns and dimensions described hereinabove can be made by micro- and nanofabrication methods known in the art. For example, bio-surface chemistry combined with micro contact printing by photolithography can be used to generate the combinatorial patterns to which a solution of the extracellular matrix proteins is added. The extracellular matrix proteins are selectively adsorbed by the micro-patterned regions to provide a substrate having a micro-patterned geometry coated with extracellular matrix proteins. Such methods are described for example by Agheli et al. (2006) Nano Lett 6:1165-71, Seidlits et al. (2008) Nanomedicine 3:183-199 and in the examples hereinbelow.

In addition, the substrates can be fabricated using nanomaterials such as nanowires, nanofibers and microwalled carbon nanotubes (MW CNTs). For example, substrates can be fabricated using MW CNT network patterns by applying CNT monolayer coatings to biocompatible polymer substrates such as polyimide, followed by selective adsorption of extracellular matrix proteins onto the CNT patterns. Such methods are described for example by Rao et al (2003) Nature 425:36-37, Park et al. (2007) Adv. Mater. 19:2530-2534, and in the examples hereinbelow.

The substrates having the micro-patterned coating of extracellular matrix proteins may then be cultured with suspensions of NSCs, which selectively adhere to the patterned regions. If desired, the adherent NSCs may be maintained in a undifferentiated state by using conditions known in the art, for example by culturing in the growth factors epidermal growth factor (EGF) and basic fibroblast growth factor (bFGF). The NSCs may be induced to differentiate to neurons by methods known in the art, for example by culturing in media without EGF and bFGF, and optionally with addition of soluble cues such as Brain Derived Neurotropic Factor, sonic hedgehog, retinoic acid, and neuropathiazol.

In another embodiment, the present invention provides methods for promoting the differentiation of NSCs to neurons comprising contacting NSCs with a substrate having one or more extracellular matrix proteins disposed thereon in geometric patterns and dimensions suitable to promote the differentiation of NSCs. NSCs are multipotent cells capable of differentiating into neurons and glial cells. In a preferred embodiment, the NSCs are mammalian NSCs. In another preferred embodiment, the NSCs are human NSCs. NSCs are commercially available or may be obtained from mammalian neural tissue by methods known in the art. This method may be used to generate differentiated cells such as neurons which are useful for methods of regeneration of neural tissue.

In another embodiment, the present invention provides a biocompatible implant comprising a substrate having one or more extracellular matrix proteins disposed thereon in geometric patterns and dimensions suitable to promote the differentiation of neural stem cells. In another embodiment, the implant further comprises at least one NSC. The implant may comprise the substrate, or the substrate contained in an implantable device, including for example a scaffold, matrix or tube.

In another embodiment, the present invention provides methods of treating or ameliorating a neurodegenerative disorder or a neurological injury comprising administering an effective amount of the biocompatible implant of the present invention to a subject in need of such treatment. Neurodegenerative disorders and neurological injuries include conditions of neuronal cell death or compromise, and include acute and chronic disorders of the central and peripheral nervous system. Such disorders and injuries include, without limitation, traumatic brain injury, spinal cord injury, peripheral nerve trauma, Alzheimer's Disease, Parkinson's Disease, Huntington's Disease, epilepsy, stroke and dementias. The implant can be delivered to a site in the central or peripheral nervous system in proximity to an area of damaged neural tissue by methods known in the art, for example by injection, infusion, or implantation. The implant may be delivered simultaneously with, before, or after another agent including for example, a drug for neural therapy, an anti-inflammatory agent, anti-apoptotic agent, or growth factor.

The present invention provides, in another embodiment, compositions comprising the substrates of the invention and a suitable carrier and compositions comprising the implants of the invention and a suitable carrier. The composition can be a pharmaceutical composition that contains a pharmaceutically acceptable carrier. The term “pharmaceutical composition” refers to the combination of an active agent with a carrier, inert or active, making the composition especially suitable for diagnostic or therapeutic use in vivo or ex vivo. The carrier in the pharmaceutical composition must be acceptable in the sense that it is compatible with the active ingredient and capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

The present invention also provides kits for use in the differentiation of NSCs and treatment of neurodegenerative disorders and neurological injuries. Such kits include at least a first container containing a composition comprising the substrate described above in a carrier. The kits may additionally contain solutions or buffers for affecting the delivery of the first composition. The kits may further contain additional containers which contain compositions comprising further agents for treatment of neurodegenerative disorders and neurological injuries including for example, a drug for neural therapy, an anti-inflammatory agent, anti-apoptotic agent, or growth factor. The kits may further contain catheters, syringes or other delivering devices for the delivery of one or more of the compositions used in the methods of the invention. The kits may further contain instructions containing administration protocols for the therapeutic regimens.

All references cited herein are incorporated herein in their entireties.

The following non-limiting examples serve to further illustrate the present invention.

### Example 1

### Materials and Methods

Synthesis and characterization of passivation molecule EG₄-(CH₂)₁₁—SH.

The procedure was adopted and modified from Lee et al. (2005) Langmuir 21(23), 10311-10315 and Derda et al. (2007) Langmuir 23(22), 11164-11167.

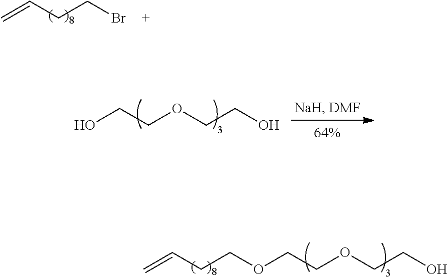

Tetraethyleneglycol (29.7 g, 153 mmoles) was dissolved in 75 ml of dry dimethylformamide under nitrogen. This solution was cooled to 0° C. and NaH (1.22 g of 60% in mineral oil, 30.6 mmoles) was added in portions. After stirring at room temperature for one hour, 7.5 g of undecenyl bromide (30.6 mmoles of 95% purity) was added and the reaction was stirred at room temperature overnight. The reaction was then diluted with 75 ml of water and extracted with 4×50 ml of hexane. The combined extracts were then washed with 2×20 ml of water, 20 ml of saturated brine, dried over MgSO₄, filtered and the solvent evaporated in vacuo. The crude product thus obtained was chromatographed on silica gel eluting with 2:1, 1:2 hexane/ethyl acetate and then with 100% ethyl acetate. The product was a light-yellow oil weighing 7.1 g (64%). The proton NMR was consistent with the desired product.

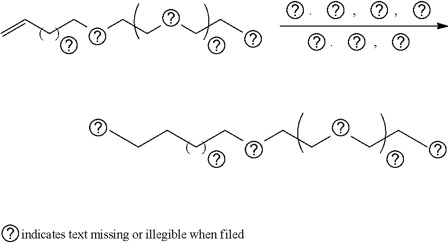

The alkene obtained previously (7.1 g, 20.5 mmoles) and thiolacetic acid (6.24 g, 82.0 mmoles) were dissolved in 75 ml of THF and this solution was deoxygenated with nitrogen. After the addition of 50 mg of AIBN, it was irradiated with 254 nm UV light overnight. The solvent was then removed in vacuo and the residue was evaporated with 3×50 ml of toluene to remove thiolacetic acid. The crude product so obtained was chromatographed on silica gel eluting with 2:1, 1:1, 1:2 hexane/ethyl acetate and finally with 100% ethyl acetate. The intermediate thioacetate product was a colorless liquid weighing 7.2 g. The proton NMR was consistent with the desired product.

The intermediate obtained above (3.0 g, 7.10 mmoles) was dissolved in a mixture of 1.5 ml of conc. hydrochloric acid and 30 ml of 95% EtOH which had been deoxygenated with nitrogen and this solution was refluxed under nitrogen overnight. It was then cooled to room temperature and the solvent removed in vacuo. The residue was partitioned between 30 ml of saturated NaHCO₃ solution and 30 ml of ethyl acetate. The phases were separated and the aqueous phase was extracted with 2×30 ml of ethyl acetate. The combined extracts were dried over MgSO₄, filtered and the solvent removed in vacuo. The product was a yellow liquid weighing 2.6 g (96%). The proton NMR was consistent with the desired product. MS: M⁺ 380.9

Generating ECM Protein Patterns.

Polycrystalline Au films were prepared by thermally depositing 5˜6 nm thick Ti layer followed by 10˜20 nm Au deposition (1.5 cm×1.5 cm) on cover glass substrates (Fisher No. 1) under a high vacuum condition (base pressure ˜5×10⁻⁶ torr). For micro contact printing, the molded PDMS stamps were prepared by conventional photolithography and 5 mM 1-octadecanethiol (ODT) was used as ink molecule. The various patterns were designed using AutoCAD so as to incorporate more multiple geometries having varying dimensions on each stamp. Pattern dimensions and spacings for each of the geometries (stripes, squares, and grids) ranged from 10 μm to 250 μm. After patterning the ODT SAMs on the thin gold films, the background was passivated using EG₄-(CH₂)₁₁—SH, a protein resistant thiol. The ECM proteins (from Sigma) such as laminin (10 μg/ml), collagen (50 μg/ml), and fibronectin (50 μg/ml) and their combinations were adsorbed on the ODT SAMs by incubating the protein solution of the SAMs for 3 h at room temperature. The protein micropatterns were then rinsed with sterile phosphate buffer saline pH 7.4 (PBS) multiple times and 2 ml suspensions of NSCs were seeded with density of 7.5×10⁴/ml (in basal medium) in a 6-well plate. The samples were incubated for 30 min. at 37° C. and each culture well containing the samples was washed gently with the NSC basal medium to remove the NSCs weakly attached on the substrate. It was observed that laminin provided the most optimum microenvironment for the adhesion and growth of the NSCs, hence all the experiments were carried out with laminin as the ECM protein. The laminin micropatterns were confirmed using anti-laminin.

Rat Neural Stem Cell (NSC) Culture and Differentiation.

Rat neural stem cell lines (Millipore) were purchased and routinely expanded according to the manufacture's protocol. The NSCs were maintained in laminin (Sigma, 20 μg/ml) coated culture dishes precoated with poly-L-lysine (10 μg/ml) in DMEM/F-12 media (Invitrogen) supplemented with B-27 (Gibco) and containing L-Glutamine (2 mM, Sigma), and antibiotics penicillin and streptomycin (Invitrogen) in the presence of basic fibroblast growth factor (bFGF-2, 20 ng/ml, Millipore). All the cells were maintained at 37° C. in a humidified atmosphere of 5% CO₂. For consistency, the experiments were carried out on the cells between passages 2 and 5. Neural differentiation was initiated by changing the medium to basal medium (without bFGF-2) on the laminin micropatterns. The cells were allowed to differentiate for 6 days with the basal medium in each being exchanged every other day.

Immunocytochemistry.

To investigate the extent of neuronal differentiation, at Day 6, the basal medium was removed and the cells fixed for 15 minutes in Formalin solution (Sigma) followed by two PBS washes. Cells were permeabilized with 0.1% Triton X-100 in PBS for 10 minutes and non-specific binding was blocked with 5% normal goat serum (NGS, Invitrogen) in PBS for 1 hour at room temperature. To study the extent of neuronal differentiation the primary mouse antibody against TuJ1 (1:500, Covance) and primary mouse antibody against Synapsin (1:500, Santa Cruz Biotechnology) were used and for glial differentiation the primary rabbit antibody against GFAP (1:500, Dako) was used. The fixed samples were incubated overnight at 4° C. in solutions of primary antibodies in PBS containing 10% NGS. After washing three times with PBS, the samples were incubated for 1 h at room temperature in a solution of anti-mouse secondary antibody labelled with cy3 and anti-rabbit secondary antibody labelled with cy2 (1:400, Jackson ImmunoResearch), Hoechst (1:500, Invitrogen) in PBS containing 10% NGS to observe neuronal and glial differentiation. After washing the samples thrice with PBS the substrates were mounted on glass slides using ProLong Gold antifade (Invitrogen) to minimize quenching by gold. To confirm that the NSCs on the micropatterns were undifferentiated at Day 2, the cells were similarly fixed and immunostained with primary and secondary antibodies. The primary rabbit polyclonal antibody against neural stem cell marker, nestin (1:400, Santa Cruz Biotechnology, Inc) was used. The secondary anti-rabbit antibody used was labelled with cy5 (1:400, Jackson ImmunoResearch). Phalloidin (1:75, Invitrogen) labelled with Alexa-546 was added to the secondary antibody solution to observe the actin cytoskeleton and alignment of the NSCs along the patterns. The mounted samples were imaged using Ziess. ImageJ (NIH) was used for comparative analysis of fluorescence signals of TuJ1 on the various pattern geometries of different dimensions, and on control samples (only laminin, no patterns).

### Example 2

### Fabrication of Extracellular Matrix Protein Patterns

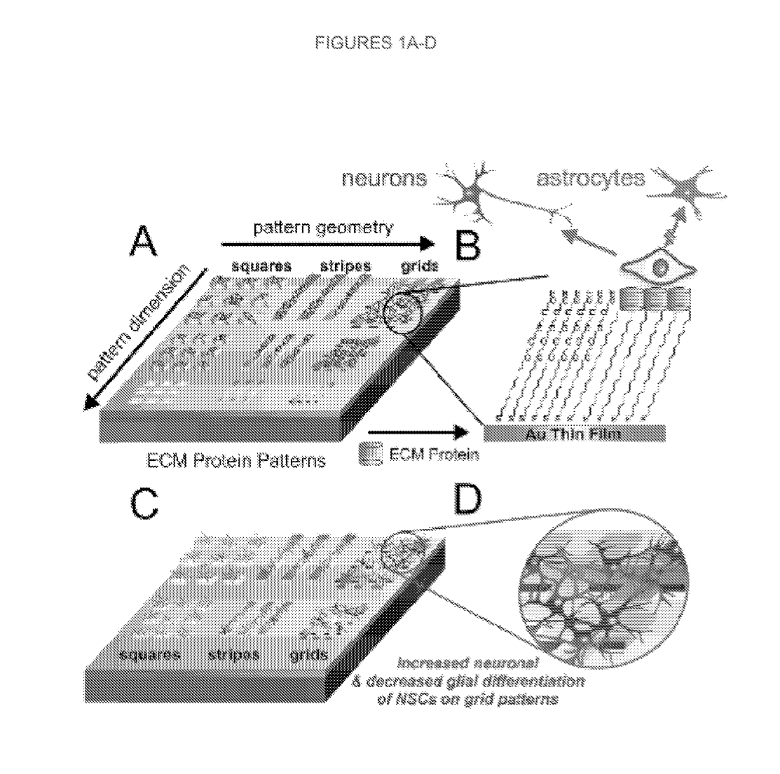

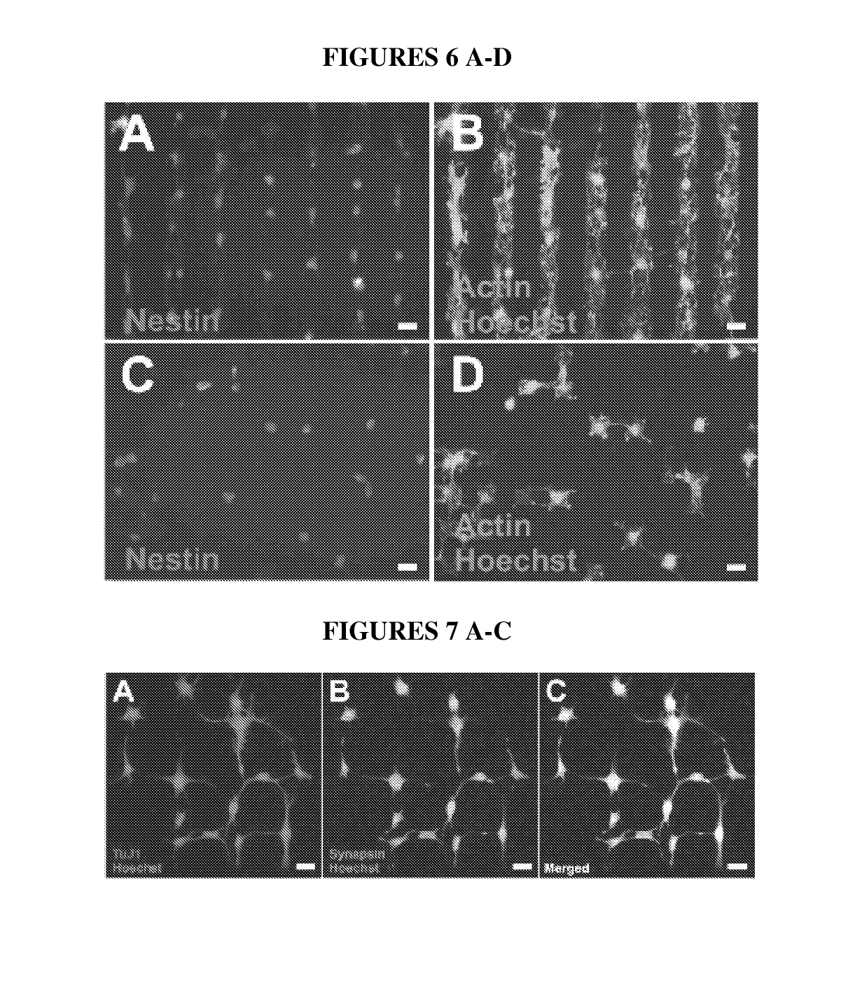

Extracellular matrix protein patterns with variant geometries and dimensions were fabricated by initially patterning octadecanethiol (ODT, 5 mM in ethanol), a hydrophobic alkanethiol, which formed self-assembled monolayers (SAMs) of squares, stripes, and grids on glass substrates coated with a thin film (12 nm) of gold. In order to minimize the nonspecific attachment of laminin, the background of the substrates was passivated by incubating in a solution (5 mM in ethanol) of tetraethylene glycol terminated alkanethiol [EG₄-(CH₂)₁₁—SH, 12 h]. After passivating the background, a solution of ECM protein [e.g. laminin (10 μg/ml) in phosphate buffered saline (PBS) buffer, pH=7.4] was added onto the substrates (3 h) and was preferentially adsorbed onto the hydrophobic regions (ODT patterns). The selective adsorption of laminin on hydrophobic regions was confirmed by immunostaining using anti-laminin IgG (FIG. 5). Only the patterned regions, coated with ECM proteins, promoted cell adhesion and growth whereas the rest of the substrate remained inert (FIG. 1). Several different ECM proteins including fibronectin and collagen were similarly patterned. Laminin provided the optimum microenviromental cues for NSC adhesion and growth. Hence, differentiation studies were carried out using laminin patterns.

### Example 3

### Effect of ECM Protein Patterns on Stem Cell Differentiation

To examine the effect of the ECM protein patterns on stem cell differentiation, primary rat hippocampal neural stem cells (NSCs) (Millipore) were first expanded and maintained in an undifferentiated state in a homogeneous monolayer on a polyornithine and laminin-coated Petri dish in a defined serum-free growth medium [DMEM/F12 supplemented with B27 and basic fibroblast growth factor (bFGF, 20 ng/ml)]. For obtaining reproducible and consistent results, all experiments were carried out using NSCs from passages 2˜5 at a constant cell density of 150,000 cells per substrate (1.5 cm×1.5 cm), which was optimum for cell growth without clustering. Arresting the proliferation of NSCs and initiating their spontaneous differentiation was achieved by withdrawing bFGF from the culture medium (resulting in basal medium), without the additional treatment with exogenous factors (proteins and small molecules). The basal medium (2 mL) containing the NSCs (75,000 cells/ml) was put in a single well of a 6-well plate containing a substrate with laminin patterns. After the NSCs attached onto the laminin patterns (1 hr), the substrates were rinsed with copious amounts of media in order to minimize non-specific interactions of NSCs with the passivated areas, and then incubated in fresh basal medium. The media was exchanged with fresh media every other day. During the screening approach to investigate the function of physical cues on neuronal differentiation of NSCs, the differentiation on ECM protein patterns was monitored by using two orthogonal assays, namely immunocytochemical and morphological assays. To assess the differentiation of NSCs, the down-regulation of the NSC marker (Nestin) and the geometry dependent expression of the neuronal marker (β-III Tubulin, TuJ1) and glial marker (glial fibrillary acidic protein, GFAP) were monitored. In addition, the development of branches or spindle-like morphologies, and neurite outgrowths were observed by using an inverted phase contrast microscope (Zeiss Axiovert 200M equipped with AxioCam CCD).

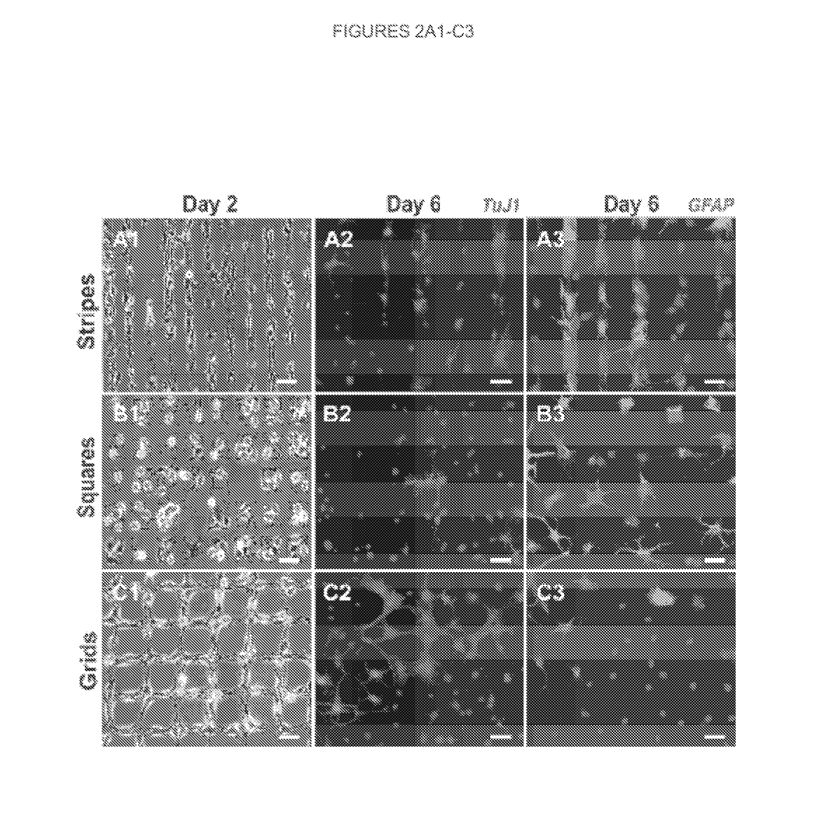

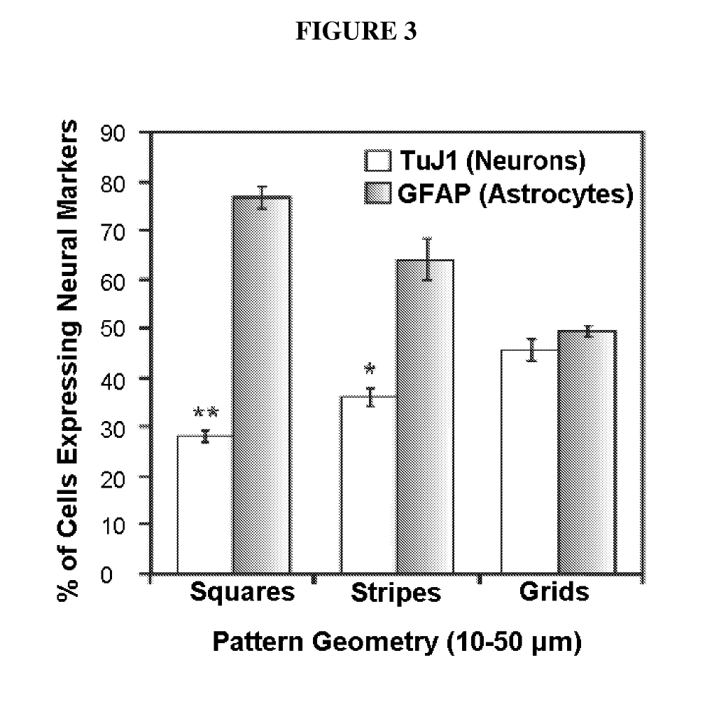

Patterns of ECM proteins with different geometries contributing to adhesion, proliferation, growth and migration of various cells (including stem cells) have been reported. (Nakajima et al. (2007) Biomaterials 28:1048; Ruiz et al. (2008) Biomaterials 29:4766; Knoll et al. (2007) Nature Protocols 2:1216.) In addition, reports from the literature have shown cell-cell interactions to play a role in the differentiation of adult stem cells. For instance, it was recently shown that cell-cell interactions played a role in the osteogenic (bone) differentiation of MSCs. (Tang et al. (2010) Biomaterials 31:2470.) To study the influences of pattern geometries and cell-cell interactions on the differentiation of NSCs, the NSCs were initially patterned on stripes of laminin, which promoted one-way interactions in a controlled manner (FIG. 2.A1). After six days, 36% of NSCs on the isolated stripes differentiated into neurons (FIG. 2.A2 and FIG. 3) and 64.3% of NSCs on these stripes differentiated into astrocytes (FIG. 2.A3 and FIG. 3).

To further confirm the influence of such interactions on the differentiation of NSCs, square patterns of laminin were used to isolate NSCs and restrict their growth within the square patterns (FIG. 2.B11). It was observed that NSCs patterned on squares, having the same dimensions and spaces as the stripes, differentiated into neurons to a considerably lesser extent (28.1%, FIG. 2.B2 and FIG. 3) as compared to the NSCs involved in one-way interactions on the striped laminin patterns. At the same time, the number of NSCs that differentiated into astrocytes increased considerably on −76.9% on squares as compared to 64.3% on stripes (FIG. 2.B3 and FIG. 3). Thus, the reduced cell-cell interactions with the NSCs on the surrounding patterns may have led to reduced neuronal differentiation and increased glial differentiation of the NSCs. To determine whether specific pattern geometries promoting cell-cell interactions could lead to higher neuronal differentiation, grid patterns of laminin, having the same dimensions as the stripe and square patterns, were used for NSC growth and differentiation. The grid patterns were specifically designed to increase cell-cell interactions in a controlled manner (by promoting two-way interactions, FIG. 2.C1). After six days in basal medium, as compared to the NSCs patterned on stripes and squares of laminin, an increase in the number of NSCs that underwent neuronal differentiation (45.6%, FIG. 2.C2 and FIG. 3) and a decrease in the number of cells that underwent glial differentiation on grid patterns of laminin (49.6%, FIG. 2.C3 and FIG. 3) were observed. All the experiments were repeated several times under the same conditions. To maintain consistency and minimize the effects from other variables, PDMS stamps were fabricated and used to generate ECM protein patterns of all the three geometries (having the same dimensions and spacing) on the same substrate. Using this method, the results were reproduced and confirmed. Neuronal and glial differentiation of NSCs was also monitored on control substrates which included substrates coated with laminin (unpatterned) and substrates without laminin. The NSCs on substrates without laminin did not attach and failed to survive, whereas 32.5% of the NSCs on the unpatterned substrates coated with laminin differentiated into neurons and 71.2% of the NSCs differentiated into astrocytes six days after seeding.

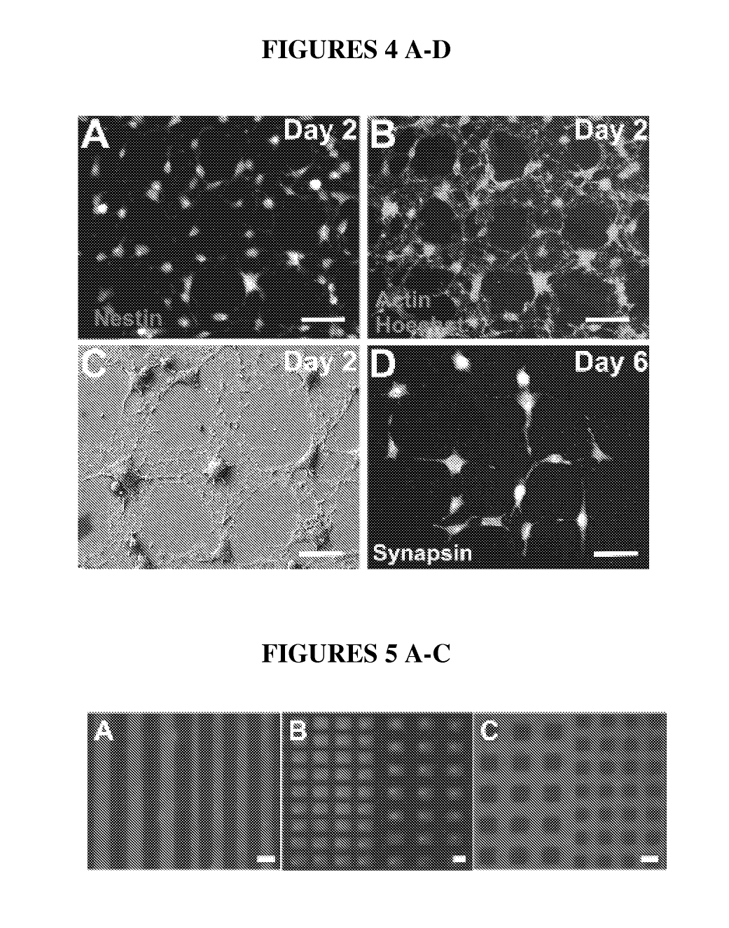

In addition to investigating the effect of pattern-geometry, the effect of dimensions on NSC differentiation was investigated. Ten different dimensions were generated for each of the geometries, ranging from sizes as small as 10 μm and as large as 250 μm (FIG. 4B). For the three different geometries above 50 μm, little difference was observed in the percentage of NSCs undergoing neuronal and glial differentiation. The result observed for pattern dimensions above 50 μm was similar to that observed with unpatterned substrates. Since the NSCs showed remarkable difference in differentiation on patterns ranging from 10-50 μm, all statistical analysis and investigation was done using pattern features within this range.

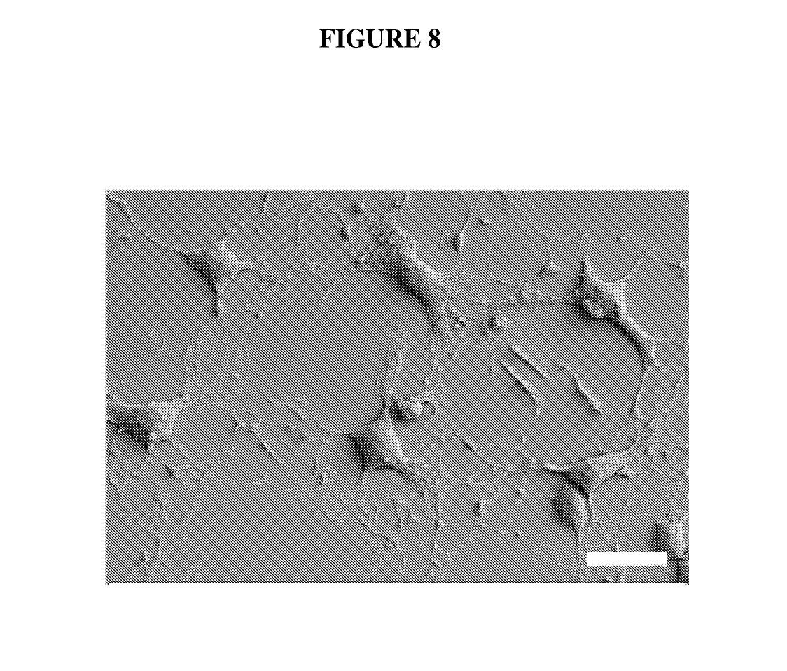

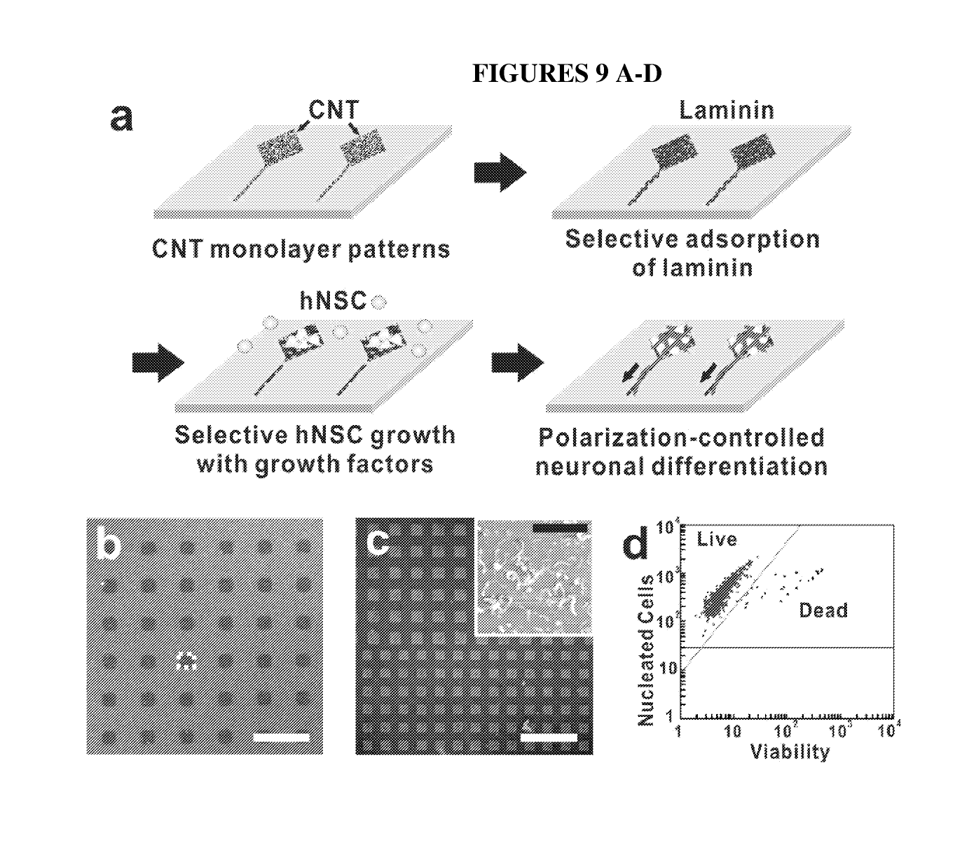

The laminin patterns of all three geometries enabled the NSCs to attach and grow within a day or two day after seeding. By staining for actin using phalloidin and using field emission scanning electron microscopy (FESEM, Zeiss Gemini), it was observed that the cytoskeleton of the NSCs aligned well within the laminin patterns, guiding cellular morphology and interactions (FIGS. 41 and C). To confirm that the laminin patterns influenced morphological changes before differentiation (as opposed to an early differentiation of NSCs which might have caused a change in alignment and morphology), the NSCs were immunostained for the neural stem cell marker nestin two days after seeding in basal medium. It was observed that most of the NSCs that aligned along the patterns stained positive for nestin (FIG. 4A), confirming that cells were in an undifferentiated (multipotent) state when they align along the patterns (See FIGS. 6A-D for NSCs on squares and stripes stained for actin and nestin). Neuronal differentiation of NSCs on the laminin patterns was further confirmed using synapsin as another neuronal marker in addition to TuJ1. After six days in basal medium, a remarkably high number of the NSCs growing along the grid patterns of laminin expressed synapsin (FIG. 4D). In addition, colocalization of TuJ11 and synapsin was observed within the NSCs differentiated on the grid patterns, confirming that the neurons expressed both neuronal markers (FIGS. 7A-C).

### Example 5

### Materials and Methods for Patterns of Carbon Nanotube Monolayer Coating

Fabrication of CNT Monolayer Pattern.

Polycrystalline Au films were prepared by thermally depositing 5˜6 nm thick Ti layer followed by 10˜20 nm Au deposition on cover glass substrates under a high vacuum condition (base pressure ˜5×10⁻⁶ torr). For micro contact printing, polydimethylsiloxane (PDMS) stamps were fabricated using photoresist (AZ5214) patterns as a template. 1-octadecanethiol (ODT, Sigma, Mo., USA) solution (5 mM in acetonitrile) was utilized as an ink. For CNT assembly, typical concentration of multi-walled CNT (Nanolab Inc, 98% purified) suspension of 0.2 mg/ml was prepared, and the ODT patterned surface by microcontact printing was placed in CNT suspension usually for 10˜30 sec and rinsed thoroughly with 1,2-dichlorobenzene. Pt (VTEC™ Polyimide 1388, Richard Blaine International, Inc., PA, USA) in solution was coated on a cover glass by spin coating at 1,000 rpm for 1 min. After the PI on cover glass was cured on a hot plate at 110° C. for 30 min in N₂ gas environment, the temperature of the hot plate was increased from 110 to 220° C. with a temperature ramping rate of 5° C./min, and then maintained at 220° C. for 2 hours. Afterwards, Ti (5˜6 nm) and Au (10˜20 nm) were thermally deposited on the PI surface.

hNSC Culture.

Immortalized human NSCs (ReNcells, Millipore, Temecula, Calif., USA) were purchased and maintained according to the manufacturer's protocol. Neural differentiation was initiated by removal of growth factors such as basic fibroblast growth factor (bFGF) and epidermal growth factor (EGF) from the culture media, and the cells were allowed to differentiate usually for two weeks. For the hNSC culture, the prepared CNT patterns were incubated in laminin solution (20 μg/ml, Sigma, Mo., USA) for 30 min. The laminin-coated CNT patterns were washed with PBS by several times and subsequently seeded with suspensions of hNSC at a cell density of 1×10⁵/ml. All the hNSC experiments were carried out between passages 3 and 10.

Cell Viability Assay.

The hNSCs were either grown for three days or subsequently differentiated for three days, and then were used for the cell viability assay. The NSCs were first detached and made into 1×10⁶/ml cell suspensions, of which only a fraction were used for counting cell viability. The cells were incubated with a reagent composed of a mixture of a cell permeant and a non-cell permeant dye (ViaCount Reagent, Millipore, Heyward, Calif., USA) according to the manufacturer's protocol and the viability was determined using a single-laser four-color flow cytometry detection system (EasyCyte Plus, Millipore, Heyward, Calif., USA) at 500 cells per one flow rate with predefined gating.

Immunocytochemistry. The hNSCs were fixed for 15 min in 4% paraformaldehyde in PBS and permeabilised with 0.1% Triton X-100 in PBS for 15 min, followed by overnight incubation at 4° C. in the following primary antibodies: TUJ1 (1:500; clone SDL.3D10, Sigma, Mo., USA), GFAP (1:1000; Dako, Glostrup, Denmark), NF-L (1:200; Millipore, Temecula, Calif., USA), GAP 43 (1:200; Millipore, Temecula, Calif., USA) and synaptophysin (Millipore, Temecula, Calif., USA). Cells were washed with PBS, incubated with either goat anti-mouse FITC (1:200; Sigma, Mo., USA) or goat anti-rabbit TRITC (1:500; Sigma, Mo., USA), then counterstained with 10 mM Hoechst 33342 (Sigma, Mo., USA). The mounted samples were imaged using an inverted fluorescence microscopy (Nikon, TE2000, Tokyo, Japan) with EMCCD monochrome digital camera (DQC-FS, Nikon, Tokyo, Japan). ImageJ software (freely downloadable from National Institutes of Health website, http://rsbweb.nih.gov/ij/) was used for subsequent processing of the fluorescence images.

### Example 6

### CNT Network Patterns

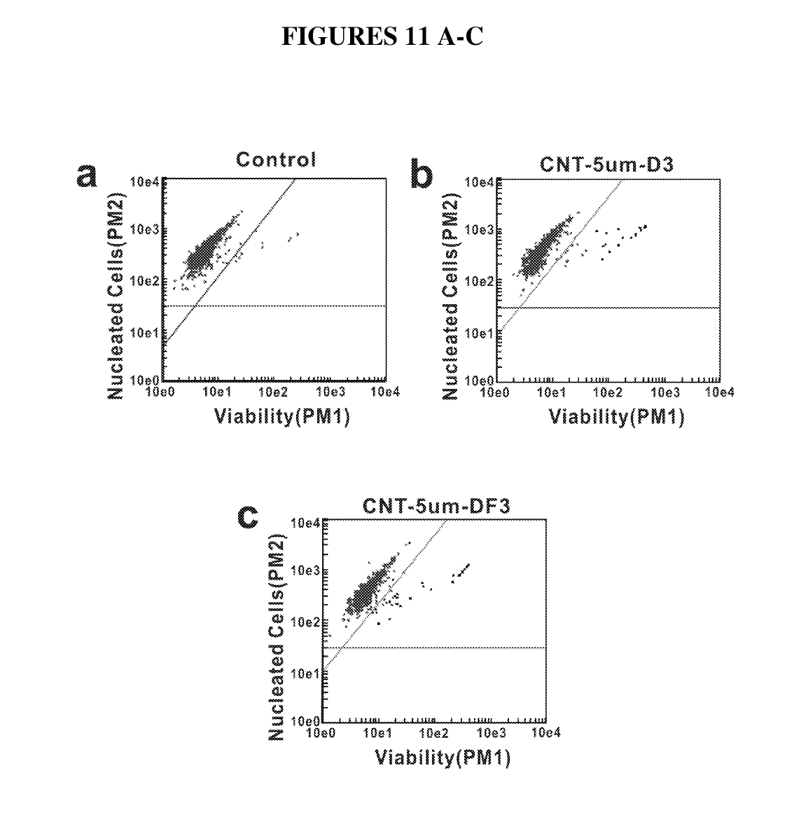

FIG. 9a shows a schematic diagram illustrating the following experimental procedure. CNT patterns were prepared according to previously-reported methods (Park et al. (2007) Adv. Mater. 19:2530-2534. Briefly, self-assembled monolayer (SAM) of methyl-terminated I-octadecanethiol (ODT) was first patterned on thin Au films on cover glass substrates by microcontact printing, while leaving some bare Au surface regions unaltered. When the patterned substrate was placed in CNT suspensions (0.5 mg/ml in 1,2-dichlorobenznene), CNTs were selectively adsorbed onto bare Au regions forming CNT monolayer patterns. The CNT patterns were then placed in laminin solution (10-20 μg/ml) for 10-30 min so that laminin molecules were selectively adsorbed onto the CNT patterns. Laminin is one of the ECM components helpful for hNSC adhesion and growth. After cell seeding, the hNSCs grew preferentially along these laminin-coated CNT patterns in the culture media with growth factors, such as basic fibroblast growth factor (bFGF) and epidermal growth factor (EGF). For hNSCs, the growth factors (bFGF and EGF) are known to enhance hNSC growth and proliferation, while blocking its neural differentiation. Afterwards, the substrate was placed in culture media without bFGF and EGF for two weeks to study the neuronal differentiation of hNSCs on laminin-coated CNT patterns.

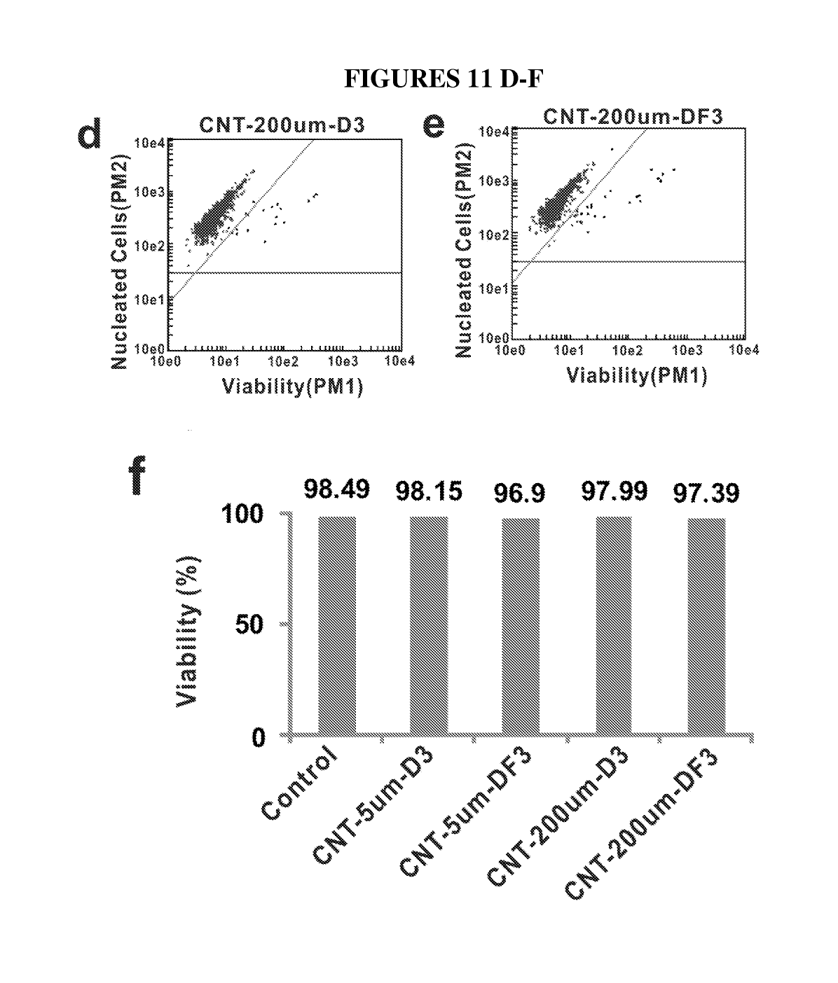

FIG. 9b shows the scanning electron micrograph (SEM) image of the prepared CNT patterns. It shows the well-defined CNT regions (darker square regions) as well as ODT-coated area (lighter region). The high resolution atomic force microscopy image also confirmed the highly-selective adsorption of CNTs on bare Au regions (FIG. 10).

When placed in laminin solution, CNT patterns induce the selective adsorption of laminin molecules. This was verified by immunochemistry (FIG. 9c). For this purpose after the laminin adsorption, the substrate was placed in the fluorescent-labeled anti-laminin solution so that the anti-laminin molecules would bind to the laminin molecules on the substrate. The fluorescence image shows much stronger fluorescence intensity in the CNT regions (brighter green regions on FIG. 9c) than on ODT regions, confirming the high-density adsorption of laminin molecules on the CNT patterns. The CNT patterns with laminin coating were also investigated via AFM topography image (insert in FIG. 9c and FIG. 10b). It exhibited the average roughness of 26 nm, which is in the optimal range of surface roughness (20˜50 nm) promoting the adhesion and longevity of primary neurons. This result indicates that the nanotopographic cues of CNT network structures as well as laminin molecules adsorbed on the CNT patterns can synergistically induce the selective growth of hNSCs.

### Example 7

### Biocompatibility of CNT Network Structures

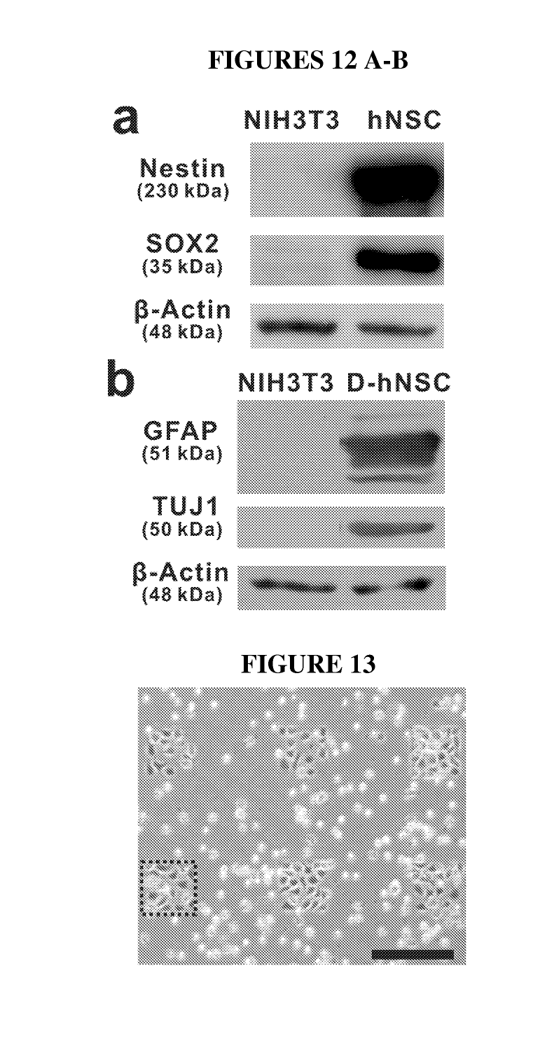

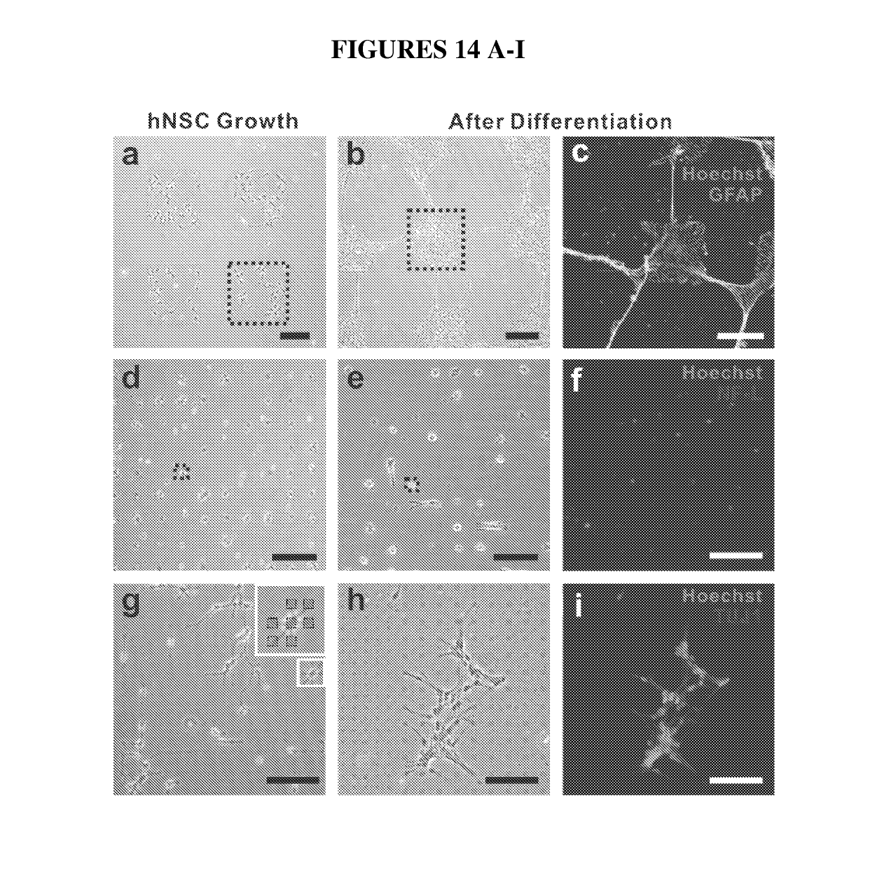

The biocompatibility of CNT network structure as a substrate for hNSC growth was investigated via cell viability assay using flow cytometry. For the assay, the adherent hNSCs were detached from the CNT patterns after three-day growth and three-day differentiation period, respectively. After the three-day growth period, 98% of the cells were found to be viable as shown in the graph (FIG. 9d). The assay result of the three-day differentiation also exhibited nearly 97% of cell viability (FIG. 11). This indicates the good biocompatibility of CNT patterns for hNSC growth and differentiation. Furthermore, the Western Blot method was utilized to confirm the protein expression of hNSCs before and after the differentiation (FIG. 12). The results show that the hNSCs grown with the growth factors (EGF and bFGF) were positive for neural stem cell markers (nestin and SOX2), which shows that they just proliferated and did not differentiate. Meanwhile, those grown without these growth factors were positive for glial fibrillary acidic protein (GFAP) and neuron-specific class Ill beta-tubulin (TUJ1), which indicates they differentiated.

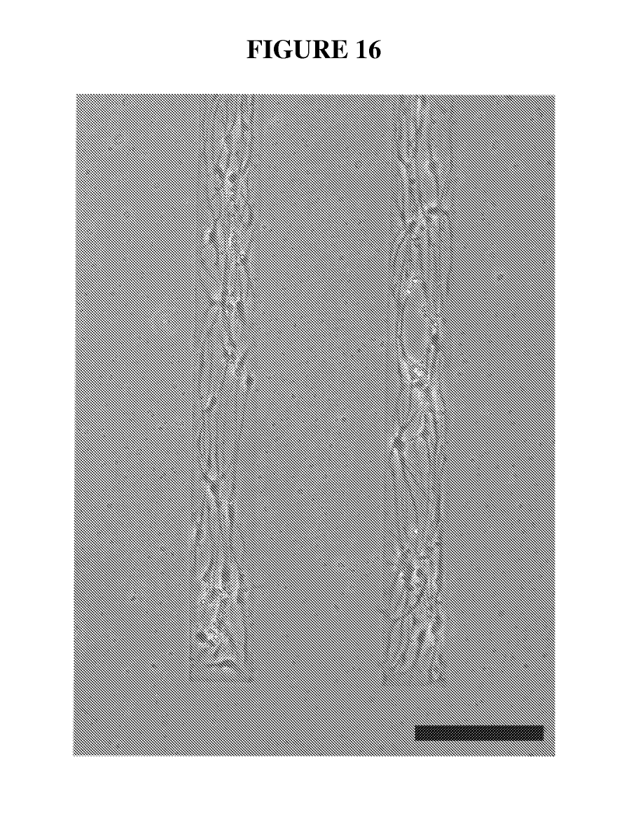

When the hNSCs were seeded on the laminin-coated CNT patterns in the culture media with the growth factors, they were selectively adhered onto the CNT pattern regions and grew along the patterns (FIG. 14a,d,g). In this stage, the growth factors blocked the neuronal differentiation of hNSCs. When the substrate was placed in the culture media without the growth factors, the hNSCs started to differentiate FIG. 14b,e,h). The neuronal differentiation was confirmed by immunocytochemistry (FIG. 14c,f,i). Here, three different markers were used to look at neural cytoskeletal distributions on the CNT patterns after the differentiation: GFAP as an astroglial cell marker (FIG. 14c), and neurofilament light (NF-L, FIG. 14f) and TUJ1 (FIG. 14i) as neuronal cell markers. An experiment was also performed to investigate the hNSC growth on CNT networks compared with that on conventional substrates such as coverglass. After the hNSC seeding on the laminin-coated CNT patterns that were prepared on coverglass, it was observed that the hNSCs grew selectively in the CNT regions. This result clearly indicates that the CNT network can provide a better extracellular environment for hNSC growth than conventional cell-culture substrates such as coverglass.

### Example 8

### Geometries of CNT Patterns

Depending on the geometries of CNT patterns, the hNSCs exhibited significantly different outgrowing behaviors during the growth and differentiation (FIG. 14). When the size of the CNT square patterns was large enough (300 μm×300 μm, 200-μm-spacing) to hold multiple cells, the hNSCs in the CNT patterns could maintain their cell-cell interactions and proliferated very well (FIG. 14a). Eventually, they outgrew over the 200-μm-wide ODT SAM regions toward the adjacent CNT square patterns and formed the neural networks, where the cells grown on the distanced CNT square patterns were connected (FIG. 14b). The fluorescence image clearly shows that the outgrowing astrocytes (green regions marked as GFAP) were connecting the hNSC population on the distanced patterns after differentiation (FIG. 14c).

The size of CNT square patterns (50 μm×50 μm, 50-μm-spacing) was then reduced such that each square could hold only a single hNSC (FIG. 14 d-f). In this case, the hNSC outgrowth was extremely restricted during the growth and differentiation process (FIG. 14d). Even after the differentiation, the hNSCs did not exhibit any indication of major outgrowth over the ODT regions (FIG. 14e). The fluorescence image of neuronal cytoskeletons (NF-L, red) does not show any outgrowing hNSCs from the patterns (FIG. 14f). This result clearly shows that the cell-cell interaction can be controlled by the geometries of CNT patterns, which can be critical for the hNSC growth and differentiation.

The hNSC behaviors on CNT square patterns smaller (5 μm×5 μm, 5-μm-spacing) than individual hNSCs (FIG. 14 g-i) was also tested. Here, the hNSCs first adhered and outgrew over several CNT square patterns (FIG. 14g). Each cell was bound strongly on the small CNT pattern regions and outgrew and extended over the ODT regions. After the differentiation, the neuronal outgrowths that extended and bound on the nearby CNT patterns (FIG. 14h) could be observed. The fluorescence image clearly showed the neuronal cytoskeletal marker (TUJ1, green) indicating the connected neural networks bound on the small CNT patterns. Overall, the results in FIG. 14 clearly show that the size and spacing of CNT patterns can play a critical role in controlling the hNSC outgrowths during the growth and differentiation process, which can possibly affect cell-cell interactions or cytoskeletal tensions.

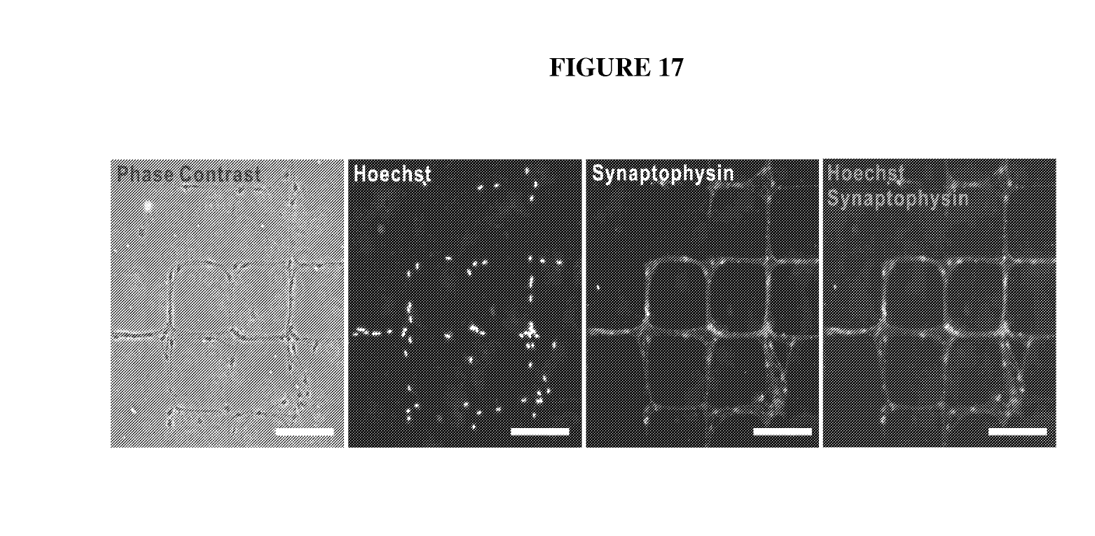

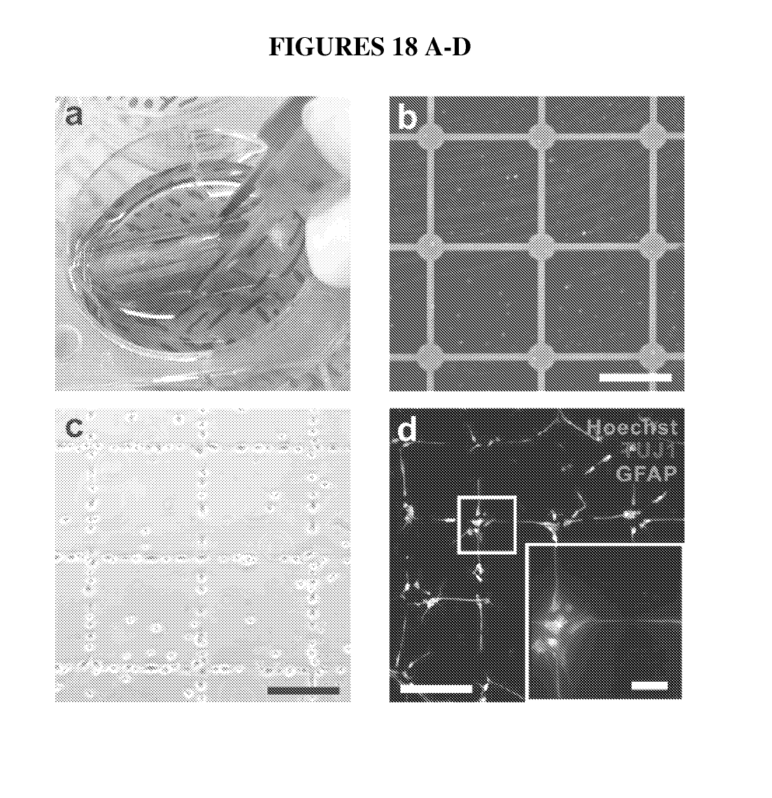

Furthermore, line-shape CNT patterns can be utilized to control the neuronal orientation with high precision (FIG. 15). In the line-shape CNT patterns with a line width (30-μm width, 60-μm-spacing) which can hold two or three cells, the hNSCs adhered (FIG. 15a) along the line pattern. It was observed that they differentiated to form neural networks along inside the line patterns (FIGS. 15b, 16). Here, the neuronal differentiation was also confirmed by immunocytochemistry with the TUJ1 marker. When the CNT line width was narrowed down to about 5 μm, which can hold only a single hNSC, the hNSCs grew and differentiated into bipolar neurons along the individual CNT line patterns (FIG. 15f). This result indicates that the orientation of neurons can be controlled with a single-cell-level precision.

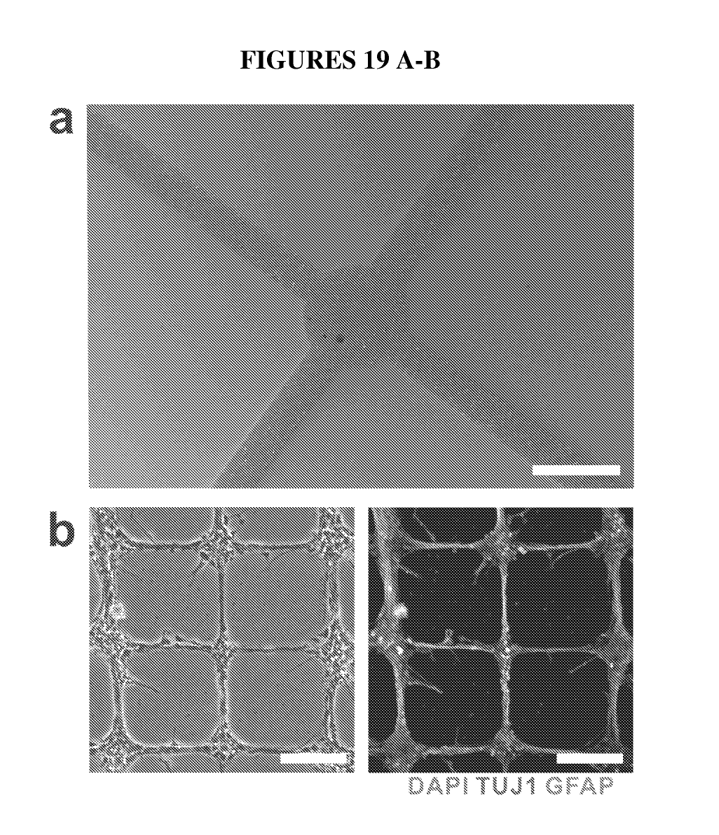

When circle-shape patterns are connected with narrow-line-shape ones, an interesting hNSC behavior was observed during the growth and differentiation (FIG. 15g-i). After seeding in the culture media with growth factors, the cell bodies of hNSCs tended to adhere and proliferated on the circle-shape pattern regions (FIG. 15g). After withdrawal of the growth factors in the culture media, they started to differentiate and the outgrowing neurites were observed mostly along the narrow line-shape CNT pattern regions (FIG. 15h). The neuronal differentiation was also confirmed via immunostaining (FIG. 15i). Since the hNSCs first adhered to grow on the circle-shape patterns, their nuclei (blue regions) were mostly located on the circle regions, while the long neurites (red regions) extended along the line-shape regions (FIG. 15i). This result indicates that the CNT patterns can be utilized to control both of the locations of cell nuclei and the direction of neurite growth, thus allowing control of the polarization of the neuronal differentiation of hNSCs. Furthermore, the synapse formation of the neurons was checked by a neuronal presynaptic vesicle marker, synaptophysin (FIG. 17). The results clearly show that the neurons differentiated from the hNSCs grown on the CNT patterns can also form the synapses, which are important for a neuron to pass a chemical/electrical signal to another neuron.

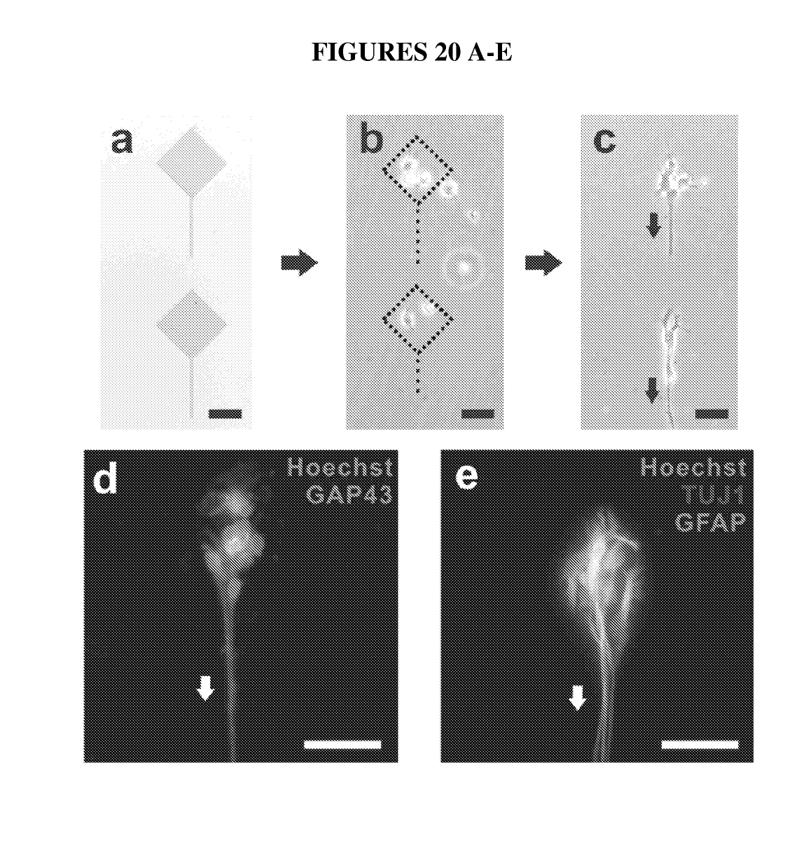

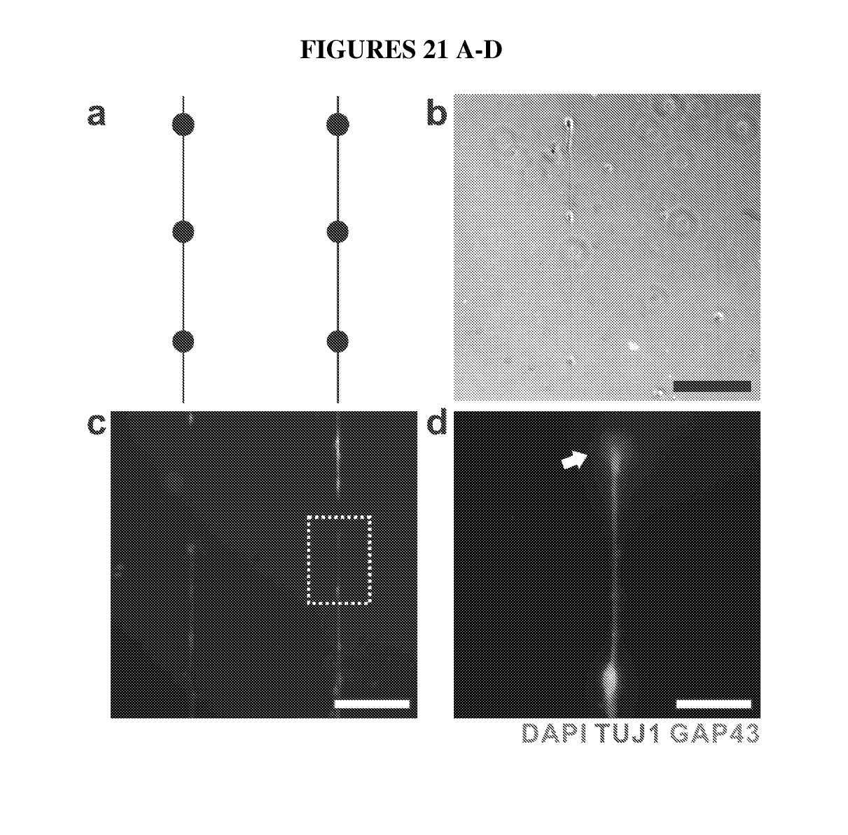

For therapeutic applications such as regenerative medicine, the foregoing strategy was applied to a flexible and biocompatible substrate such as polyimide (PI) (FIG. 18) which has been widely utilized for implantable neural devices (Schmidt et al. (2003) Annu. Rev. Biomed. Eng. 5:293-347). CNT patterns were prepared on thin-Au-film-coated PI substrates and experiments of hNSC growth and differentiation were performed (FIG. 18a). High-quality CNT patterns were achieved on the Au-coated PI substrates as shown in the SEM images (FIG. 19a). The immunofluorescence image indicated the highly-selective adsorption of laminin onto the CNT patterns on the PI substrate (FIG. 19b). After seeding on it, the hNSCs adhered selectively onto the CNT pattern regions on PI substrates and proliferated (FIG. 19c). Eventually, orientation-controlled growth and differentiation of hNSCs along the CNT patterns was achieved on the flexible PI substrate (FIGS. 18d, 19b).

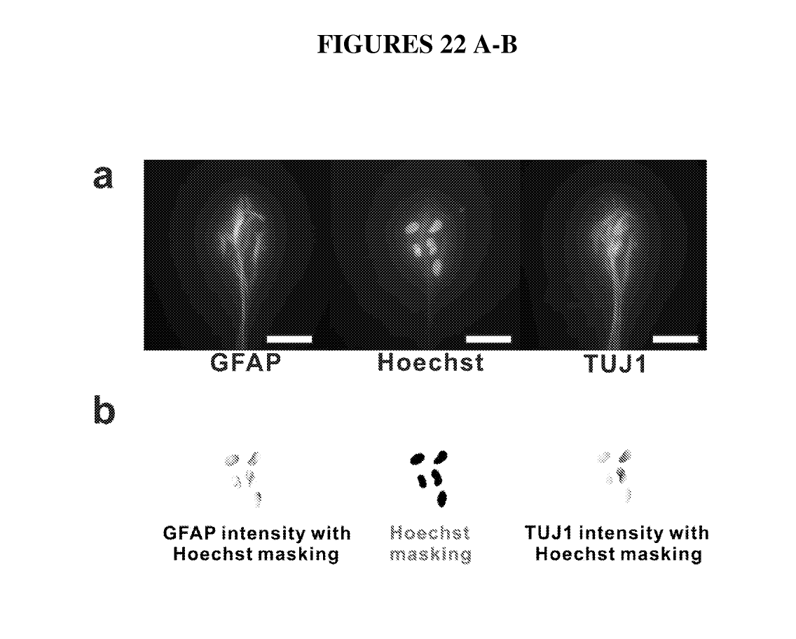

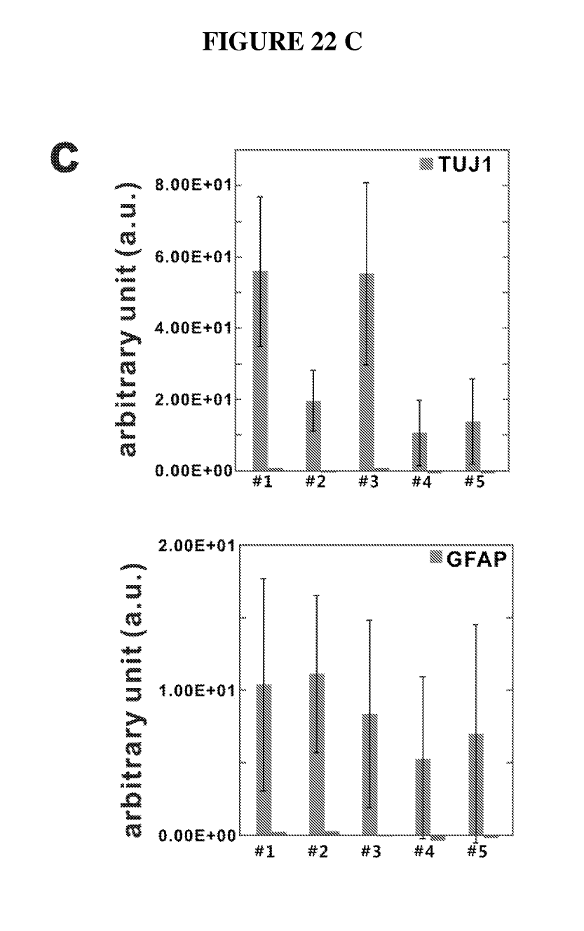

Polarization-controlled differentiation of individual hNSCs was achieved by CNT patterns comprised of one square- and one line-shape ones (FIG. 20a). Here, the width of the line-shape region is much smaller than the size of an individual hNSC. After cell seeding, selective hNSC adhesion inside the square regions was observed (FIG. 20b). The hNSCs on the square region then outgrew along the narrow line-shape regions during the growth and differentiation stages (FIG. 20c). The neuronal differentiation was confirmed by growth associated protein 43 (GAP 43, green in FIG. 20d) which is known to be expressed in the growth cone regions of neural cells. It was observed that the GAP 43 was also highly expressed on the line-shape CNT regions, indicating the neurites outgrew along the line-shape regions (FIGS. 20d, 21).

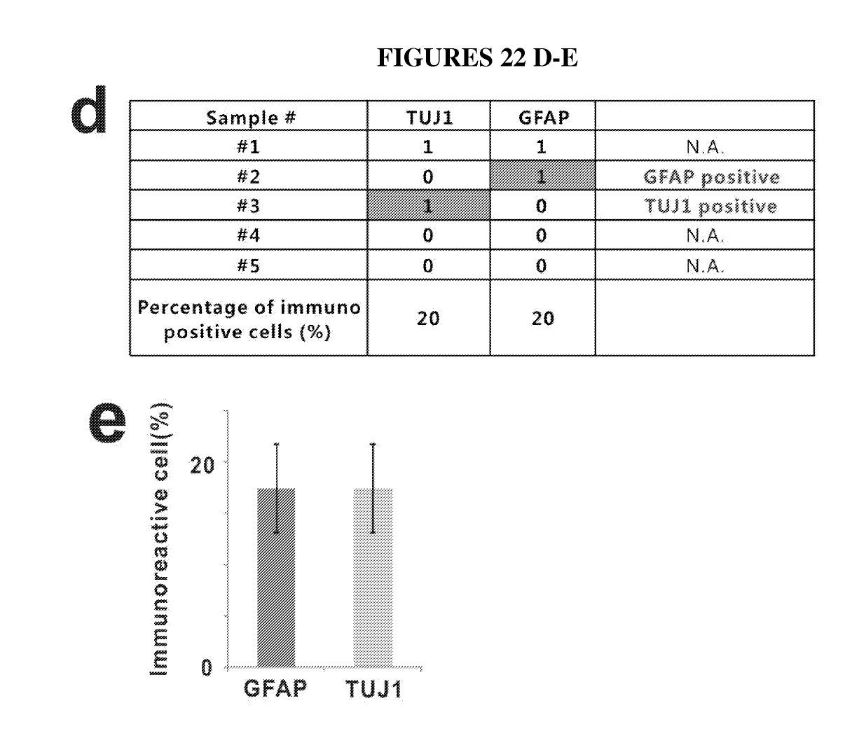

Immunocytochemistry was performed to check the neural lineages of the differentiated cells on these CNT patterns (FIG. 20e). Here, GFAP and TUJ1 indicate astroglial and neural cells, respectively. To confirm their lineages, the relative fluorescence intensities of GFAP and TUJ1 from the cell nuclei on the square pattern regions were quantified using the similar method reported previously (FIG. 22)) Soen et al. (2006) Mol. Syst. Biol. 2:37. The result showed that 20% of them were TUJ1-positive, whereas another 20% were GFAP-positive. The hNSCs were differentiated with controlled-polarity on the CNT patterns, while maintaining their capabilities to differentiate into the main phenotypes in the nervous system, such as neuronal cells and astroglial ones.

The foregoing results demonstrate a polarization-controlled neural differentiation of hNSCs using patterns of CNT network structures. Due to the synergistic effects of CNT network structures for the selective adsorption of ECM proteins and optimal nanotopography, the selective adhesion and growth of hNSC on the CNT patterns was promoted. The cell viability assay result (>97%) also indicated good biocompatibility of CNT patterns for hNSC growth and differentiation. The polarization-controlled neural differentiation was demonstrated at the level of an individual axon or neurite, was applied to flexible and biocompatible PI substrates.

What is claimed is:

1. A substrate having one or more extracellular matrix proteins disposed thereon in a regular two-dimensional geometric pattern, wherein the proteins in the pattern have an average height dimension of from about 50 nm to about 200 nm.

2. The substrate of claim 1 wherein the one or more extracellular matrix proteins comprise one or more of laminin, fibronectin and collagen.

3. The substrate of claim 1 wherein the extracellular matrix protein is laminin.

4. The substrate of claim 1 wherein the pattern is selected from stripes, squares, lines, squares connected by lines, circles connected by lines, and a grid.

5. The substrate of claim 1 wherein the substrate is biocompatible.

6. The substrate of claim 1 wherein the substrate comprises polyimide

7. The substrate of claim 1 wherein the substrate has at least one neural stem cell (NSC) in contact therewith.

8. The method of claim 7 wherein the NSC is a human NSC.

9. The substrate of claim 1, wherein the geometric pattern is provided by a carbon nanotube monolayer.

10. A method for promoting the differentiation of NSCs to neurons comprising contacting NSCs with a substrate having one or more extracellular matrix proteins disposed in a regular two-dimensional geometric pattern, wherein the proteins in the pattern have a height dimension of from about 1 μm to about 50 μm.

11. A biocompatible implant comprising a substrate having one or more extracellular matrix proteins disposed thereon in a regular two-dimensional geometric pattern, wherein the proteins in the pattern have a height dimension of from about 1 μm to about 50 μm.

12. The implant of claim 11 further comprising at least one NSC.

13. The implant of claim 11 wherein the implant is a scaffold, matrix or tube.

14. A method of treating or ameliorating a neurodegenerative disorder or a neurological injury comprising administering the biocompatible implant comprising a substrate having one or more extracellular matrix proteins disposed thereon in a regular two-dimensional geometric pattern, wherein the proteins in the pattern have a height dimension of from about 1 μm to about 50 μm and further comprising at least one NSC, to a subject in need of such treatment.

15. The method of claim 14 wherein the NSC is a human NSC.

16. A composition comprising the substrate of claim 1 and a carrier.

17. A kit comprising the composition of claim 16.
